SEQUENCE LISTING (5 sequences)

SEQ ID NO: 1 - RNA, 21 nt, Artificial Sequence
  Synthetic Sequence
  modified_base (1)..(1): Modified with Cy5
  modified_base (21)..(21): Modified with 1-O-propargyl-2-deoxy-D-furanose
cauguuguucagcugcugcuu

SEQ ID NO: 2 - RNA, 21 nt, Artificial Sequence
  Synthetic Sequence
  modified_base (21)..(21): Modified with 1-O-propargyl-2-deoxy-D-furanose
cauguuguucagcugcugcuu

SEQ ID NO: 3 - RNA, 21 nt, Artificial Sequence
  Synthetic Sequence
  modified_base (21)..(21): Modified with 1-O-propargyl-2-deoxy-D-furanose
gcagcagcugaacaacauguu

SEQ ID NO: 4 - RNA, 21 nt, Artificial Sequence
  Synthetic Sequence
  modified_base (21)..(21): Modified with 1-O-propargyl-2-deoxy-D-furanose
gcgacgaccuuacagagcguu

SEQ ID NO: 5 - RNA, 21 nt, Artificial Sequence
  Synthetic Sequence
  modified_base (21)..(21): Modified with 1-O-propargyl-2-deoxy-D-furanose
cgcucuguaaggucgucgcuu

(19) United States
(12) Patent Application Publication
(10) Pub. No.: US 2025/0319193 A1
(43) Pub. Date: Oct. 16, 2025

(54) CONJUGATE, PREPARATION METHOD THEREOF AND USE THEREOF

(71) Applicant: Cosychem Biotechnology (Tianjin) Co., Ltd., Tianjin (CN)

(72) Inventors: Zhen XI, Tianjin (CN); Chao YANG, Tianjin (CN)

(21) Appl. No.: 18/556,303

(22) PCT Filed: Apr. 21, 2022
(86) PCT No.: PCT/CN2022/088173

§ 371 Date: Oct. 19, 2023

(30) Foreign Application Priority Data
Apr. 23, 2021 (CN) 202110442394.6

(51) Int. Cl. A61K 47/64; A61K 47/54; A61K 47/60; A61P 35/00

(52) U.S. Cl. CPC A61K 47/64; A61K 47/542; A61K 47/60; A61P 35/00

(57) ABSTRACT

The disclosure relates to the field of biotechnology, discloses a conjugate, preparation method thereof and use thereof. The conjugate is formed by covalently linking an azido-modified targeting ligand to a propargyl-modified small nucleic acid sequence. The conjugate provided in the present disclosure has broad application prospects in drug targeted delivery. In addition, the present invention also provides a preparation method for the conjugate and a use thereof. The method only relates to simple chemical reactions, can achieve the purpose of flexibly and efficiently synthesizing a nucleic acid conjugate, is suitable for constructing other ligand-targeted nucleic acid conjugates, and has relatively of good practicability.

### CROSS REFERENCE TO RELATED APPLICATIONS

This application is a National Stage application of PCT/CN2022/088173, filed Apr. 21, 2022, which claims the benefit of Chinese Patent Publication No. 202110442394.6, filed Apr. 23, 2021, both of which are incorporated by reference in their entirety herein.

### SEQUENCE LISTING

The Instant Application contains a Sequence Listing which has been submitted electronically in ASCII format and is hereby incorporated by reference in its entirety. Said ASCII copy, created on Nov. 4, 2024, is named “RNP0011US_ST25” and is 1,933 bytes in size. The Sequence Listing does not go beyond the disclosure in the application as filed.

### FIELD OF INVENTION

The present disclosure relates to the field of biotechnology, in particular to a conjugate, preparation method thereof and use thereof.

### BACKGROUND

Prostate specific membrane antigen (PSMA), also known as folic hydrolase 1 (FOLH1), glutamic acid carboxypeptidase II (GCP II), N-acetyl-α-linked acid dipeptidase I (NAALAD1), belongs to the M28B subfamily of the peptidase M28 family. PSMA is a type II transmembrane protein with a homodimer structure, which is capable of binding two zinc ions per subunit. PSMA has both folate hydrolase and N-acetyl-α-linked acid dipeptidase (NAALADase) activity, which regulates the hydrolysis of N-acyl polyγ-glutamic acid and derivatives thereof. Compared with other tissues, PSMA is highly expressed in the nervous system, prostate, pancreas, kidney and small intestine, and its expression is much higher in the epithelial layer of most prostate cancers and in the neovascularization of other solid tumors than in normal tissues. This tissue specificity makes PSMA one of the most attractive targets for prostate cancer delivery.

Due to the high enrichment of PSMA in prostate cancer cells, antibodies, aptamers and small molecule ligands that specifically recognize PSMA are scrambling for research and development. The study of small molecule ligands for PSMA has been of great interest, due to the simplicity of synthesis and high protein binding affinity. As a small molecule ligand of PSMA, lysine/glutamic acid urea-linked dipeptide (KUE) has been successfully applied in prostate cancer imaging and targeted therapy. In addition, RNA interference (RNAi) is a novel technology that uses therapeutic oligonucleotides such as siRNAs to treat various diseases, including cancer. Encouraged by the approvals of Patisiran and Givosiran by Food and Drug Administration (FDA), RNAi is also gaining attention for the treatment of prostate cancer. Moreover, the approval of Givosiran makes the direct conjugate of a ligand to a siRNA a simple and effective new way for targeted cancer therapy. However, how to effectively deliver therapeutic siRNA into prostate cancer tissues/cells to achieve cancer treatment remains a significant challenge. Additionally, how to break through the limitations of the solid-phase synthesis method used by Givosiran (such as poor flexibility, complicated operation, unsatisfactory synthesis efficiency, and narrow application range and so on) is also a key problem that needs to be solved urgently.

### SUMMARY

The object of the present disclosure is to overcome the technical problems such as poor flexibility, complicated operation, and limited production scale of production of nucleic acid conjugates existing in the prior art, and to provide a conjugate, preparation method thereof and use thereof. The conjugate is characterized by both of high targeting and high activity. The method provided by the disclosure has the advantages of rapid, efficient, simple and widely applicable as well. In addition, the present disclosure provides the use of nucleic acid conjugates prepared according to the present disclosure in the preparation of a drug for the treatment of any one of prostate cancer, colon cancer, pancreatic cancer, breast cancer, kidney disease, and nervous system related diseases, in particular the drug for the treatment of prostate cancer.

To achieve the above objects, the first aspect of the present disclosure provides a conjugate formed by an azido-modified targeting ligand covalently linked to a propargyl-modified small nucleic acid sequence.

The second aspect of the present disclosure provides a method for preparing the aforementioned conjugate, comprising making contact between an azido-modified targeting ligand and a propargyl-modified small nucleic acid sequence in the presence of a copper monovalent catalyst.

The third aspect of the present disclosure provides an use of the aforementioned conjugate or a conjugate prepared by the aforementioned method in the preparation of a drug for the treatment of a disease related to an abnormality in a tissue expressing PSMA, preferably occurring in glandular tissue, colon, kidney, and nervous system; wherein the glandular tissue is selected from one of prostate, pancreas, breast and thymus.

The present disclosure enables efficient and rapid construction of nucleic acid conjugates of the present disclosure through the ingenious design of propargyl-modified oligonucleotides (especially modified at the 3′-end) and azido-modified targeting ligands, and by utilizing the method of the present disclosure, namely post-synthesis modification strategy (copper-catalyzed click chemistry). Through cell imaging, gene silencing evaluation, and apoptosis experiments, it is found that the conjugate (nucleic acid conjugate) provided by the disclosure has high specific recognition performance, gene silencing performance, and inhibition of tumor cell generation performance. The method for preparing conjugates (nucleic acid conjugates) provided by the present disclosure has broad application prospects in targeted delivery of RNAi therapeutic reagents and therapy. In addition, the preparation method provided by the present disclosure only involves simple chemical reactions, but it can achieve flexible and efficient synthesis of nucleic acid conjugates, and is also applicable to the construction of a wild variety of other ligand-targeted nucleic acid conjugates, which is of good practicability.

### BRIEF DESCRIPTION OF THE DRAWINGS

FIG. 1 is a schematic diagram of a KUE-conjugated small nucleic acid in the present disclosure;

FIG. 2 is a high performance liquid chromatography chromatogram and a mass spectrum of 3′-terminal propargyl-modified oligonucleotide 1 (ON1) in the present disclosure;

FIG. 3 is a high performance liquid chromatography chromatogram and a mass spectrum of 3′-terminal propargyl-modified oligonucleotide 2 (ON2) in the present disclosure;

FIG. 4 is a high performance liquid chromatography chromatogram and a mass spectrum of 3′-terminal propargyl-modified oligonucleotide 3 (ON3) in the present disclosure;

FIG. 5 is a polyacrylamide gel electrophoresis characterization of different KUE-PEG-siRNA conjugates in the present disclosure;

FIG. 6 is a polyacrylamide gel electrophoresis characterization of different DUPA-PEG5-siRNA conjugates in the present disclosure;

FIG. 7 is a mass spectrum of the nucleic acid conjugate KUE-PEG2-siRNA in Example 1 of the present disclosure;

FIG. 8 is a mass spectrum of the nucleic acid conjugate KUE-PEG5-siRNA in Example 21 of the present disclosure;

FIG. 9 is a mass spectrum of the nucleic acid conjugate KUE-PEG12-SiRNA in Example 22 of the present disclosure;

FIG. 10 is a mass spectrum of the nucleic acid conjugate DUPA-PEG5-siRNA in Example 23 of the present disclosure;

FIG. 11 is a mass spectrum of the nucleic acid conjugate DUPA-PEG5-siRNA in Example 24 of the present disclosure;

FIG. 12 is a diagram which shows the cell imaging results of different nucleic acid conjugates in one specific embodiment of the present disclosure;

FIG. 13 is a statistical chart of the mean intracellular fluorescence intensity of different nucleic acid conjugates in one specific embodiment of the present disclosure;

FIG. 14 is a diagram which shows the results of evaluation of the cellular internalization mode of different nucleic acid conjugates in one specific embodiment of the present disclosure;

FIG. 15 is an evaluation diagram of uptake efficiency of different nucleic acid conjugates in prostate cancer cells in one specific embodiment of the present disclosure;

FIG. 16 is a diagram of mRNA silencing results of different nucleic acid conjugates in one specific embodiment of the present disclosure;

FIG. 17 is a diagram of protein expression inhibition results of different nucleic acid conjugates in one specific embodiment of the present disclosure;

FIG. 18 is a diagram shows the results of apoptosis promoted by different nucleic acid conjugates in the present disclosure.

### DETAILED DESCRIPTION OF THE INVENTION

The end points and any values of the ranges disclosed herein are not limited to that precise range or value, which should be understood to include values close to those ranges or values. “For numerical ranges, one or more new numerical ranges may be combined between the endpoint values of the individual ranges, between the endpoint values of the individual ranges and individual point values, and between the individual point values, which shall be deemed to be specifically disclosed herein.

The inventors found that the azido modified targeting ligands (such as azido modified KUE ligand, azido modified DUPA ligand, etc.) and 3′-terminal propargyl modified small nucleic acid (oligonucleotide) can flexibly and efficiently construct antigen (such as PSMA) targeted siRNA conjugates with a huge number of studies. In addition, in order to verify whether 3′-terminal-propargyl-modified small nucleic acids can maintain their thermal stability, overall conformation and RNAi activity, the inventors studied the related properties of 3′-terminal-propargyl-modified small nucleic acids in aspects such as melting chain temperature, conformation and RNAi activity evaluation, and found that alkyne-modification of 3′-terminal of siRNAs not only does not affect the intrinsic properties of the RNAs, but also accentuates the properties of the post-synthesis modifications of the siRNAs.

Based on the context above, the first aspect of the present disclosure provides a conjugate formed by an azido-modified targeting ligand covalently linked to a propargyl-modified small nucleic acid sequence.

According to some embodiments of the present disclosure, the azide group in the azido-modified targeting ligand is covalently linked to the targeting ligand by at least one fragment of polyethylene glycol. Wherein, the number of PEG monomers in the PEG fragment is not specially limited, for example, it can be 1-100. Wherein, the fragment of the PEG mainly functions as a linker.

According to some embodiments of the present disclosure, the targeting ligand is a dipeptide formed by two amino acids in which at least one amino acid is glutamic acid; preferably, the amino acids are selected from lysine and glutamic acid, glutamic acid and glutamic acid, and glutamic acid and glutamate analogs; wherein, the targeting ligand is able to recognize and bind to antigens expressed on the surface of target cells through glutamate; The antigen is PSMA.

According to some embodiments of the present disclosure, the end of the targeting ligand contains a carboxyl group; the targeting ligand is linked by a carboxyl group to an antigen on the surface of the target cell.

According to some embodiments of the present disclosure, the target cell may be selected from at least one of prostate cancer cells, neuronal cells, kidney cancer cells, and colon cancer cells.

According to some embodiments of the present disclosure, the azido modified targeting ligand has a structure as follows:

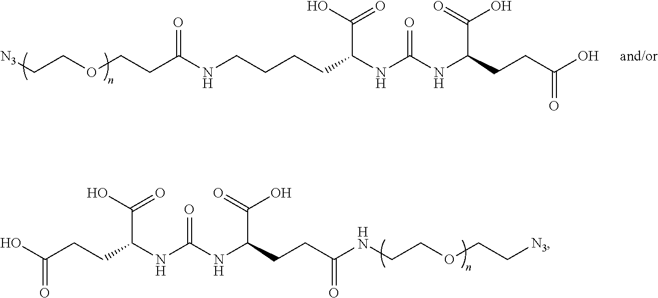

wherein n is an integer from 1 to 100. Preferably, n is an integer from 1 to 50, more preferably an integer from 1 to 20, further preferably an integer from 1 to 15. For example, it could be 1, 2, 3, 4, 5, 6, 7, 8, 9, 10, 11, 12, 13, 14, 15.

In the present disclosure, the azide-modified targeting ligand can be prepared by the following method:

Firstly, KUE was synthesized by linking lysine protected by t-butyl ester and benzyl ester with glutamate protected by t-butyl ester through triphosgene; After that, the ethylene glycol was acrylated and azided to obtain azido PEG propionic acid; Finally, the targeting ligand can be obtained by condensation reaction with both of them (KUE and azido PEG propionic acid).

In the present disclosure, preferably, the azide-modified targeting ligand (6a(n=2), 6b(n=5), 6c(n=12)) can be prepared by the following steps:

(1) The polyethylene glycol la-c was exposed to the metal sodium, followed by the first reaction with tert-butyl acrylate to obtain the compound shown in 2a-c;

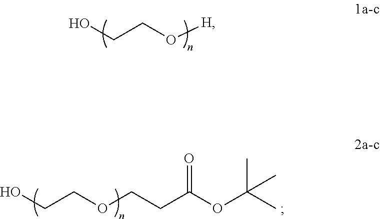

(2) In the presence of triethylamine, the compound shown in Formula 2a-c was subjected to a second reaction with tosyl chloride to obtain the compound shown in Formula 3a-c;

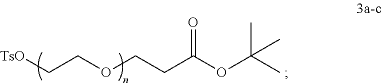

(3) In the presence of the third solvent, the compound shown in Formula 3a-c was subjected to the third reaction with sodium azide to obtain the compound shown in Formula 4a-c;

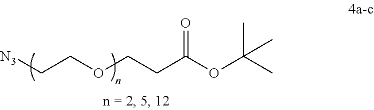

(4) In the presence of HATU (2-(7-aza-benzotriazole)-N,N,N′,N′-tetramethylurea hexafluorophosphate) and DIPEA (N,N-diisopropylethylamine), the compound shown in 4a-c was subjected to the fourth reaction with KUE to obtain the compound shown in Formula 5a-c;

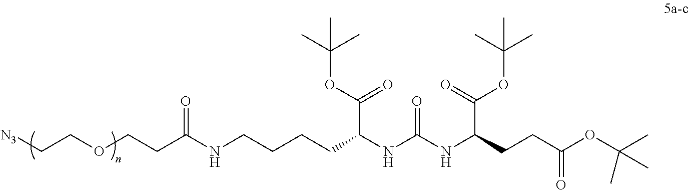

(5) The compound shown in Formula 5a-c was subjected to the fifth reaction with trifluoroacetic acid to obtain the compound shown in Formula 6a-c;

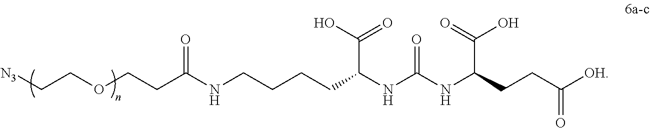

In step (1), the reaction temperature of the first reaction may be 20-40° C., and the reaction time of the first reaction may be 5-20 hours. Wherein, relative to each millimole of polyethylene glycol (1a-c), the amount of metal sodium may be 0.05-2 mmol, the amount of t-butyl acrylate may be 0.3-0.8 mmol. The first reaction is carried out in the presence of a first solvent; Wherein, relative to each millimole of polyethylene glycol, the amount of the first solvent may be 1-2 mL; The first solvent may be tetrahydrofuran.

In step (2), the reaction temperature of the second reaction may be from −20° C. to 10° C., and the reaction time of the second reaction may be 5-20 hours. Wherein, relative to each millimole of the compound shown in Formula 2a-c, the amount of triethylamine is 0.5-1 ml, and the amount of tosyl chloride may be 1.2-1.8 mmol. The second reaction is carried out in the presence of a second solvent; wherein, relative to each millimole of the compound shown in Formula 2a-c, the amount of the second solvent may be 1-3 mL. Wherein, the second solvent may be dichloromethane. The second reaction is preferably carried out under an inert atmosphere, which may be provided by argon.

In step (3), the reaction temperature of the third reaction may be 60-90° C. and the reaction time of the third reaction may be 5-24 hours. Wherein, relative to each millimole of the compound shown in Formula 3a-c, the amount of sodium azide may be 1.2-1.8 mmol. Wherein, relative to each millimole of the compound shown in Formula 3a-c, the amount of the third solvent may be 2-3 mL. The third solvent may be DMF (N,N dimethylformamide).

In step (4), the reaction temperature of the fourth reaction may be 20-40° C. and the reaction time of the fourth reaction may be 5-24 hours. Wherein, relative to each millimole of the compound shown in Formula 4a-c, the amount of KUE may be 0.9-1.5 mmol. Wherein, prior to the fourth reaction between the compound shown in 4a-c and KUE, the compound shown in 4a-c may be de-protected in the presence of trifluoroacetic acid (the amount of trifluoroacetic acid is 2-3 ml relative to 1 mol of the compound shown in 4a-c), wherein the temperature of de-protection may be 20-30° C., and the time of de-protection is 10-24 h; HATU and DIPEA may then be used to activate the de-protection products of the compound shown in 4a-c; Wherein, relative to each millimole of the compound shown in Formula 4a-c, the amount of HATU and DIPEA may be 1.2-1.8 mmol and 2-4 mmol, respectively. The fourth reaction is carried out in the presence of a fourth solvent; Wherein, relative to each millimole of the compound shown in Formula 4a-c, the amount of the fourth solvent may be 0.8-1.5 mL. The fourth solvent may be DMF (N,N dimethylformamide).

In step (5), the reaction temperature of the fifth reaction may be 20-40° C. and the reaction time of the fifth reaction may be 5-24 hours. Wherein, relative to each millimole of the compound shown in Formula 5a-c, the amount of trifluoroacetic acid may be 20-30 mL.

Wherein, there is no special restriction on the post-treatment of the reactions in steps (1)-(5), and all the purified products may be obtained by column chromatography separation.

In the present disclosure, the structural formula of the KUE is as follows:

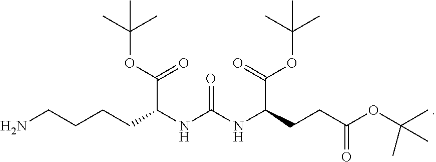 [SMILES: CC(C)(C)OC(=O)CC[C@@H](NC(=O)N[C@H](CCCCN)C(=O)OC(C)(C)C)C(=O)OC(C)(C)C]

According to some embodiments of the present disclosure, the small nucleic acid sequence may be selected from at least one of the small nucleic acids targeting STAT3, PHB1, Notch1, PLK1, and BRD4.

According to a preferred embodiment of the present disclosure, the propargyl modification is preferably a 3′-terminal propargyl modification.

According to some embodiments of the present disclosure, the alkyne-modified small nucleic acid sequence is modified to the 3′-terminal of the small nucleic acid sequence using the phosphoramidite method by solid-phase synthesis techniques with propargyl compound; Wherein, the propargyl compound contains at least one active hydroxyl group. Wherein, the propargyl modification may be more than one propargyl modifications (1, 2, 3 . . . ). In the present disclosure, there is no particular limitation on the manner in which the propargyl compound is modified to the 3′-end of the small nucleic acid sequence, it may be performed as follows: First, based on the prior art (Bioorg. Med. Chem., 2013, 5583-5588), 1-O-propargyl-2-deoxy-D-furanose phosphoramidite monomer was synthesized from commercial Hoffer's chlorosugar through four steps. Then, the resulting phosphoramidite monomer was linked to a controlled pore glass (CPG) and the 3′-terminal of the propargyl monomer X oligonucleotide was inserted by an automatic nucleic acid synthesizer; Finally, the CPG and protective group were removed to obtain 3′-terminal propargyl-modified small nucleic acids.

In the present disclosure, preferably, the propargyl compound has a structure as follows:

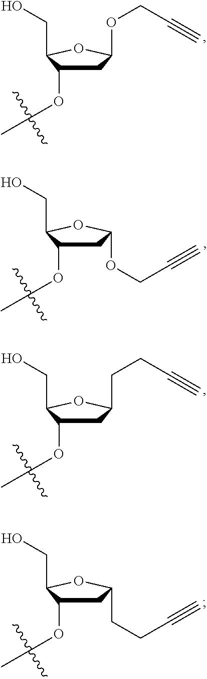

wherein“ [symbol] ” represents the small nucleic acid ligation site.

The second aspect of the present disclosure provides a method for preparing the aforementioned conjugate, comprising making contact between an azido-modified targeting ligand and a propargyl-modified small nucleic acid sequence in the presence of a copper monovalent catalyst.

According to some embodiments of the present disclosure, the molar ratio of the azido-modified targeting ligand to the propargyl-modified small nucleic acid sequence may be (1.05-10):1, preferably (2-4):1.

According to some embodiments of the present disclosure, the copper monovalent catalyst may be selected from at least one of Cu(I)-TBTA, CuBr, and CuCl, preferably Cu(I)-TBTA.

According to some embodiments of the present disclosure, relative to 1 mol of the small nucleic acid sequence, the amount of the copper monovalent catalyst may be 2-10 mol, preferably 3-6 mol.

According to some embodiments of the present disclosure, the conditions of the contact comprising: temperature may be 35.5-38.5° C., preferably 36.5-37.5° C.; the time of the contact may be 1-5 h, preferably 2-4 h.

In the present disclosure, there is no special limitation to the speed of the contact, as long as it can meet the requirements of the present disclosure, for example, the speed may be 600-1000 r/min, preferably 700-900 r/min.

In the present disclosure, the contact is preferably carried out under the condition of shock, and the condition of shock may be: once every 0.4-0.6 h.

According to some embodiments of the present disclosure, the contact is carried out in the presence of an organic solvent selected from at least one of N,N-dimethylformamide (DMF), methanol, ethanol, and acetone.

According to some embodiments of the present disclosure, the amount of organic solvent is such that the concentration of the small nucleic acid sequence is maintained between 20 and 200 μM, preferably between 80 and 120 μM.

In the present disclosure, the conjugate may refer to a single-stranded nucleic acid conjugate or a double-stranded nucleic acid conjugate; When the conjugate is a double-stranded nucleic acid conjugate, it may be formed by annealing from the sense and antisense strand nucleic acids with reference to conventional means in the art, for example, by forming the sense and antisense strand nucleic acids in equimolar proportions in an annealing buffer (containing magnesium ions) at 90-100° C.

According to some embodiments of the present disclosure, the small nucleic acid sequence is a siRNA sequence comprising a sense strand and an antisense strand, and the method comprising:
(1) Propargyl-modified sense strand and propargyl-modified antisense strand were obtained separately;
(2) Single-stranded conjugate was obtained by contacting the azide-modified target ligand with the propargyl-modified sense strand or propargyl-modified antisense strand;
(3) The conjugate was obtained by incubating the single-stranded conjugate with the other strand in annealing buffer at 90-100° C. for 1-5 minutes;

According to a preferred embodiment of the present disclosure, the annealing buffer is selected from a magnesium acetate solution of 1.5-2.5 mM.

The third aspect of the disclosure provides an use of the aforementioned conjugate or a conjugate prepared by the aforementioned method in the preparation of a drug for the treatment of a disease related to an abnormality in a tissue expressing PSMA, preferably occurring in glandular tissue, colon, kidney, and nervous system; wherein, the glandular tissue is selected from one of the prostate, pancreas, breast and thymus, wherein, the disease is preferably a cancer.

In the present disclosure, “conjugate” refers to “KUE-PEG-siRNA conjugate” or “DUPA-PEG-siRNA conjugate”.

The present disclosure will be described in detail by examples below.

In the following examples, all raw materials are commercially available.

### Preparation Example 1

This preparation example is used to illustrate the preparation of azide-modified KUE ligand 6a-c.

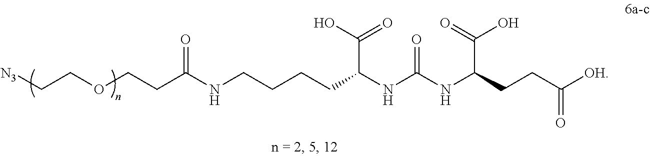

Synthetic route 1:

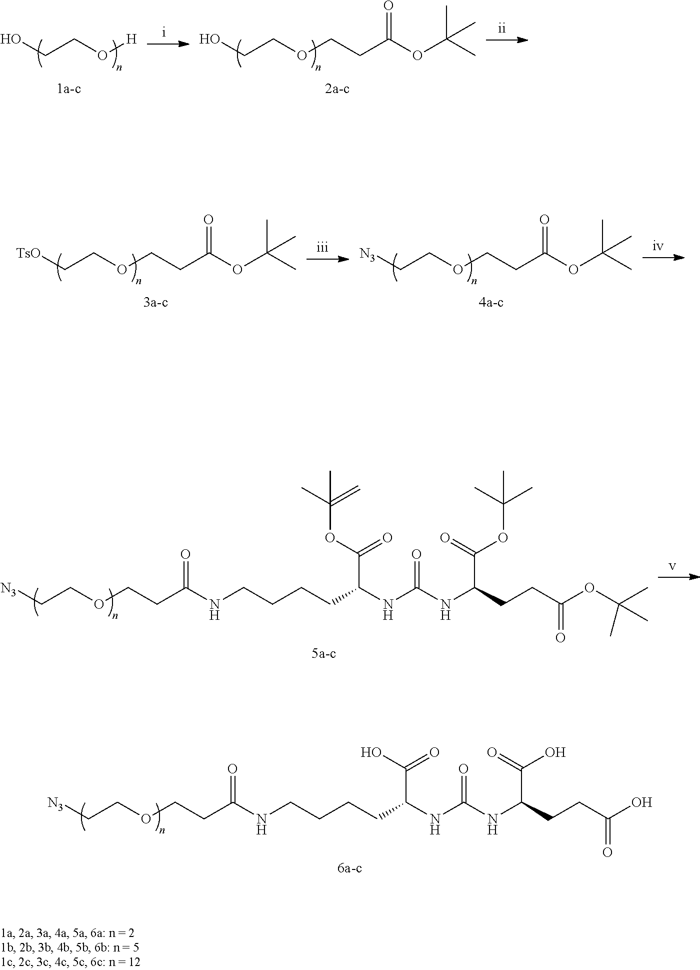

Specific operation steps:

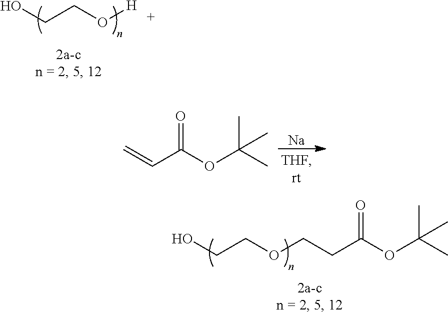

(i) Polyethylene glycosl 1a-c (100 mmol, 1 eq) were dissolved in dry THF (150 mL), to which metal sodium (230 mg, 10 mmol, 0.1 eq) was added and stirred at room temperature until the sodium was completely dissolved. Subsequently, tert-butyl acrylate (6.4 g, 50 mmol, 0.5 eq) was added slowly to the mixture and reacted for 12 hours at room temperature. At the end of the reaction, the reaction was quenched by the addition of 8 mL of 1M aqueous hydrochloric acid. After stirring for 10 minutes, the reaction solution was poured into 200 mL of saturated saline, extracted with ethyl acetate for 3 times, the organic phase was collected, washed with saturated saline, dried with anhydrous sodium sulfate, and purified by rapid column chromatography (PE/EA=10:1-1:1) to obtain a slightly yellow mono-tert-bytyl ester product.

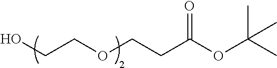

¹H-NMR (400 MHZ, CDCl₃) δ 3.77-3.70 (m, 4H), 3.69-3.59 (m, 6H), 2.78 (s, 1H), 2.54-2.49 (m, 2H), 1.45 (s, 9H); ¹³C-NMR (101 MHZ, CDCl₃) δ170.92, 80.63, 72.49, 70.33, 70.31, 66.81, 61.70, 36.14, 28.05. HRMS: calculated for C₁₁H₂₂NaO₅ [M+Na]⁺: 257.1365, found 257.1357.

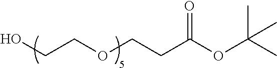

¹H-NMR (400 MHZ, CDCl₃) δ 3.79-3.59 (m, 22H), 2.91 (s, 1H), 2.53-2.48 (m, 2H), 1.45 (s, 9H); ¹³C-NMR (101 MHZ, CDCl₃) δ 170.93, 80.51, 72.52, 70.61, 70.59, 70.56, 70.54, 70.48, 70.33, 66.88, 61.72, 36.24, 28.09. HRMS: calculated for C₁₇H₃₄NaO₈ [M+Na]⁺: 389.2151, found 389.2146.

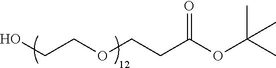

¹H-NMR (400 MHZ, CDCl₃) δ 3.76-3.54 (m, 49H), 2.77 (s, 2H), 2.53-2.48 (m, 2H), 1.45 (d, J=2.4 Hz, 9H); ¹³C-NMR (101 MHz, CDCl₃) δ 170.94, 80.53, 72.58, 70.61, 70.57, 70.51, 70.37, 70.29, 66.90, 61.71, 36.26, 28.10. HRMS: calculated for C₃₁H₆₂NaO₁₅ [M+Na]⁺: 697.3986, found 697.3981.

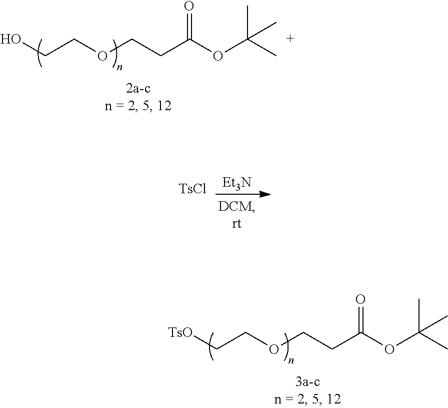

(ii) Compound 2a-c (12.3 mmol, 1 eq) was dissolved in a mixture of 30 mL dichloromethane and 10 mL triethylamine and stirred in an ice bath under an argon atmosphere. Then, p-toluenesulfonyl chloride (3.53 g, 18.5 mmol, 1.5 eq) was added in batches. The mixture was stirred overnight at room temperature, then, 10 g of a small amount of silica gel (100-200 mesh) was concentrated to dryness, and chromatographic separation (PE/EA=10:1-1:3) was performed on silica gel (300-400 mesh), obtaining a colorless oil form product.

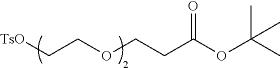

¹H-NMR (400 MHZ, CDCl₃) δ 7.79 (d, J=8.4 Hz, 2H), 7.35 (d, J=8.0 Hz, 2H), 4.19-4.11 (m, 2H), 3.67 (dd, J=8.0, 4.8 Hz, 4H), 3.55 (q, J=4.8 Hz, 4H), 2.47 (dd, J=12.4, 6.0 Hz, 5H), 1.44 (s, 9H); ¹³C-NMR (101 MHZ, CDCl₃) δ 170.81, 144.80, 132.99, 129.82, 127.95, 80.50, 70.60, 70.29, 69.26, 68.64, 66.88, 36.21, 28.07, 21.61. HRMS: calculated for C₁₈H₂₈NaO₇S [M+Na]⁺: 411.1453, found 411.1448.

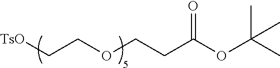

¹H-NMR (400 MHZ, CDCl₃) δ 7.85-7.74 (m, 2H), 7.37-7.33 (m, 2H), 4.24-4.08 (m, 2H), 3.83-3.51 (m, 20H), 2.59-2.36 (m, 5H), 1.45 (d, J=5.6 Hz, 9H); ¹³C-NMR (101 MHZ, CDCl₃) δ 170.89, 144.78, 133.02, 129.82, 127.98, 80.49, 70.74, 70.58, 70.51, 70.36, 69.24, 68.67, 66.89, 36.27, 28.09, 21.64. HRMS: calculated for C₂₄H₄₀NaO₁₀S [M+Na]⁺: 543.2240, found 543.2246.

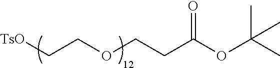

¹H-NMR (400 MHZ, DMSO) δ 7.83-7.74 (m, 2H), 7.49 (d, J=7.6 Hz, 2H), 4.13-4.10 (m, 2H), 3.64-3.29 (m, 48H), 2.43 (s, 5H), 1.40 (s, 9H); ¹³C-NMR (101 MHZ, DMSO) δ 170.87, 145.34, 132.88, 130.59, 128.10, 80.15, 70.44, 70.25, 70.17, 70.14, 68.35, 66.69, 36.29, 28.20, 21.55. HRMS: calculated for C₃₈H₆₈NaO₁₇S [M+Na]⁺: 851.4075, found 851.4071.

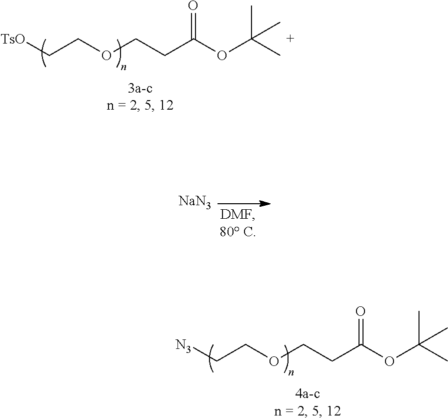

(iii) Compound 3a-c (2 mmol, 1 eq) was dissolved in 5 mL of dimethylformamide and sodium azide (195 mg, 3 mmol, 1.5 eq) was added. The mixture was allowed to react overnight at 80° C., and then concentrated to dryness by adding 2 g of a small amount of silica gel (100-200 mesh) and chromatographic separation (PE/EA=20:1-1:2) was performed on silica gel (300-400 mesh), resulting in a colorless oil form product.

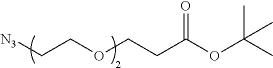

¹H-NMR (400 MHZ, CDCl₃) δ 3.76-3.60 (m, 8H), 3.42-3.35 (m, 2H), 2.51 (t, J=6.4 Hz, 2H), 1.45 (s, 9H); ¹³C-NMR (101 MHz, CDCl₃) δ 170.84, 80.45, 70.56, 70.38, 70.00, 66.92, 50.64, 36.22, 28.04. HRMS: calculated for C₁₁H₂₁N₃NaO₄ [M+Na]⁺: 282.1430, found 282.1426.

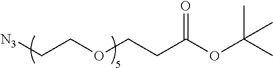

¹H-NMR (400 MHZ, CDCl₃) δ 3.77-3.58 (m, 20H), 3.47-3.30 (m, 2H), 2.50 (t, J=6.8 Hz, 2H), 1.45 (s, 9H); ¹³C-NMR (101 MHz, CDCl₃) δ 170.92, 80.51, 70.70, 70.67, 70.64, 70.59, 70.50, 70.37, 70.04, 66.90, 50.68, 36.26, 28.09. HRMS: calculated for C₁₇H₃₃N₃NaO₇ [M+Na]⁺: 414.2216, found 414.2211.

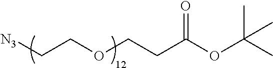

¹H-NMR (400 MHZ, CDCl₃) δ 3.75-3.57 (m, 48H), 3.40 (d, J=4.4 Hz, 2H), 2.53-2.48 (m, 2H), 1.45 (d, J=2.4 Hz, 9H); ¹³C-NMR (101 MHz, CDCl₃) δ 170.91, 80.49, 70.69, 70.66, 70.63, 70.56, 70.49, 70.36, 70.04, 66.88, 50.67, 36.25, 28.09. HRMS: calculated for C₃₁H₆₁N₃NaO₄ [M+Na]⁺: 722.4051, found 722.4048.

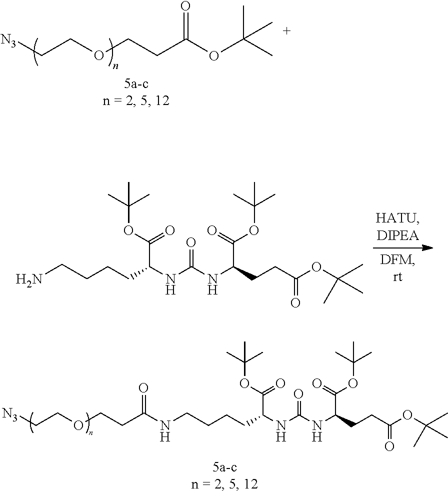

(iv) Compound 4a-c (2 mmol, 1 eq) was dissolved in 5 mL of trifluoroacetic acid and was allowed to react overnight at room temperature. Then, the remaining trifluoroacetic acid was removed by concentration, dichloromethane and 2 g silica gel (100-200 mesh) were added and concentrated to dryness, and chromatographic separation (PE/EA=5:1-1:5) was carried out on silica gel (300-400 mesh), resulting a product as colorless oil, which was dissolved in 2 mL of dimethylformamide. HATU (1.15 g, 3 mmol, 1.5 eq) and DIPEA (775.5 mg, 6 mmol, 3 eq) were added for activation. After 15 min, KUE (1.17 g, 2.4 mmol, 1.2 eq) was added. The mixture was allowed to react overnight at room temperature, then, 3 g of small amount of silica gel (100-200 mesh) was concentrated to dryness, and chromatographic separation (PE/EA=10:1-1:5) was performed on silica gel (300-400 mesh), obtaining the product in colorless oil form.

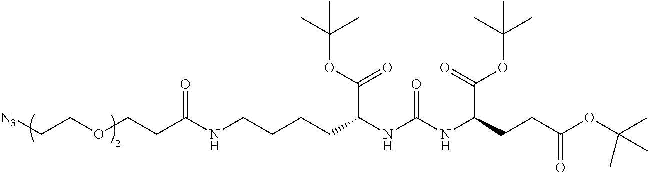

¹H-NMR (400 MHZ, CDCl₃) δ 6.87 (s, 1H), 5.64 (dd, J=20.0, 6.4 Hz, 2H), 4.38-4.17 (m, 2H), 3.71 (d, J=28.0 Hz, 7H), 3.44 (s, 2H), 3.34-3.12 (m, 2H), 2.52 (s, 2H), 2.32 (d, J=5.2 Hz, 2H), 2.06 (d, J=6.0 Hz, 1H), 1.84 (dd, J=32.0, 25.2 Hz, 2H), 1.67-1.23 (m, 33H); ¹³C-NMR (101 MHz, CDCl₃) δ 172.58, 172.52, 157.40, 82.17, 81.66, 80.67, 70.19, 70.00, 69.76, 67.35, 53.35, 52.99, 50.56, 38.84, 36.42, 32.02, 31.55, 28.63, 28.04, 27.96, 22.23. HRMS: calculated for C₃₁H₅₆N₆NaO₁₀ [M+Na]⁺: 695.3956, found 695.3953.

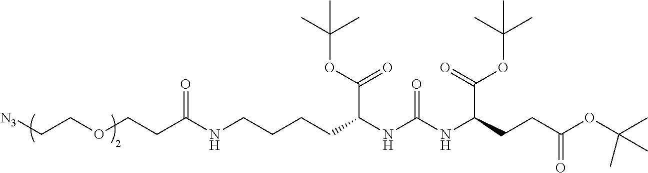

¹H-NMR (400 MHZ, CDCl₃) δ 7.05 (s, 1H), 5.78 (dd, J=16.4, 8.0 Hz, 2H), 4.38-4.17 (m, 2H), 3.71 (d, J=29.2 Hz, 15H), 3.41 (d, J=4.0 Hz, 1H), 3.26 (dd, J=23.2, 17.2 Hz, 2H), 3.00 (s, 3H), 2.50 (s, 1H), 2.32 (d, J=6.4 Hz, 2H), 2.05 (s, 1H), 1.91-1.70 (m, 2H), 1.69-1.17 (m, 36H); ¹³C-NMR (101 MHZ, CDCl₃) δ 172.21, 161.56, 157.58, 157.41, 81.74, 81.19, 80.34, 70.47, 70.45, 70.41, 70.37, 70.35, 70.28, 70.18, 70.01, 69.87, 67.24, 53.35, 52.83, 50.54, 45.11, 39.74, 38.74, 36.60, 32.17, 31.92, 31.47, 29.03, 28.70, 28.20, 27.97, 27.91, 22.55, 22.31. HRMS: calculated for C₃₇H₆₈N₆NaO₁₃ [M+Na]⁺: 827.4742, found 827.4738.

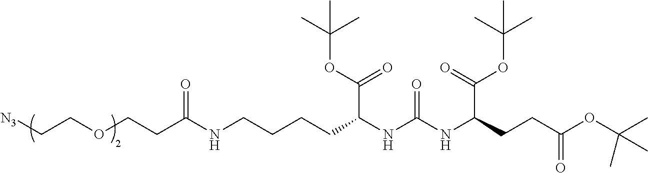

¹H-NMR (400 MHZ, CDCl₃) δ 6.89 (s, 1H), 5.57 (s, 2H), 4.26 (d, J=20.4 Hz, 2H), 3.67 (s, 45H), 3.41 (s, 2H), 3.21 (d, J=16.8 Hz, 2H), 2.99 (s, 1H), 2.53 (s, 1H), 2.32 (s, 2H), 2.05 (s, 1H), 1.80 (d, J=44.4 Hz, 1H), 1.65-1.18 (m, 36H); ¹³C-NMR (101 MHZ, CDCl₃) δ 172.41, 157.42, 81.87, 81.44, 80.50, 70.37, 70.21, 70.05, 69.99, 69.82, 69.71, 69.62, 69.49, 69.37, 53.47, 53.02, 50.61, 38.87, 31.97, 31.55, 28.20, 28.06, 27.98, 22.31. HRMS: calculated for C₅₁H₉₆N₆NaO₂₀ [M+Na]⁺: 1135.6577, found 1135.6569.

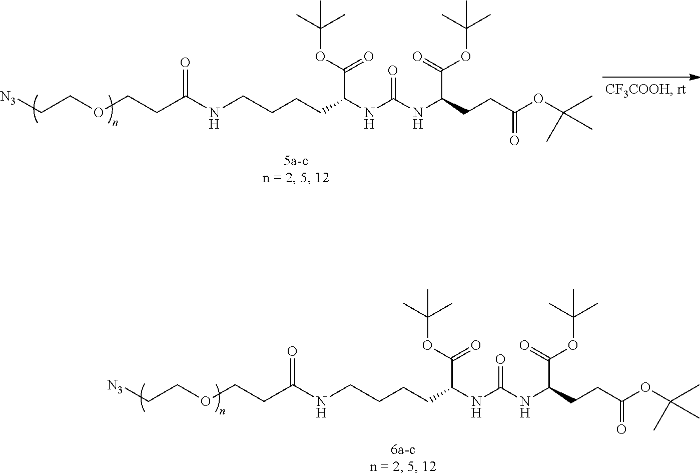

(v) Compound 5a-c (0.2 mmol, 1 eq) was dissolved in 5 mL of trifluoroacetic acid and was allowed to react overnight at room temperature. Then, the remaining trifluoroacetic acid was removed by concentration, dichloromethane and 1 g of small amount of silica gel (100-200 mesh) were added and concentrated to dryness, and chromatographic separation (DCM/MeOH=10:1-1:2) was carried out on silica gel (300-400 mesh), obtaining the product in colorless oil form. The compounds shown in 6a-c were confirmed by high-resolution mass spectrometry.

6a: 145 mg, Yield 96%, HRMS: calcd. for C₁₉H₃₂N₆O₁₀ [M−H]⁻: 503.2102; MALDI-TOF-MS: m/z 503.2105;

6b: 179 mg, Yield 94%, HRMS: calcd. for C₂₅H₄₄N₆O₁₃ [M−H]⁻: 635.2888; MALDI-TOF-MS: m/z 635.2892;

6c: 260 mg, Yield 92%, HRMS: calcd. for C₃₉H₇₂N₆O₂₀ [M−H]⁻: 943.4723; MALDI-TOF-MS: m/z 943.5601.

### Preparation Example 2

This preparation example is used to illustrate the preparation of azide-modified DUPA ligands

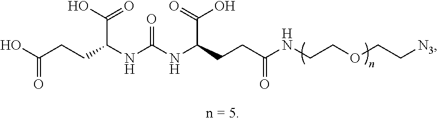

Synthetic route 2:

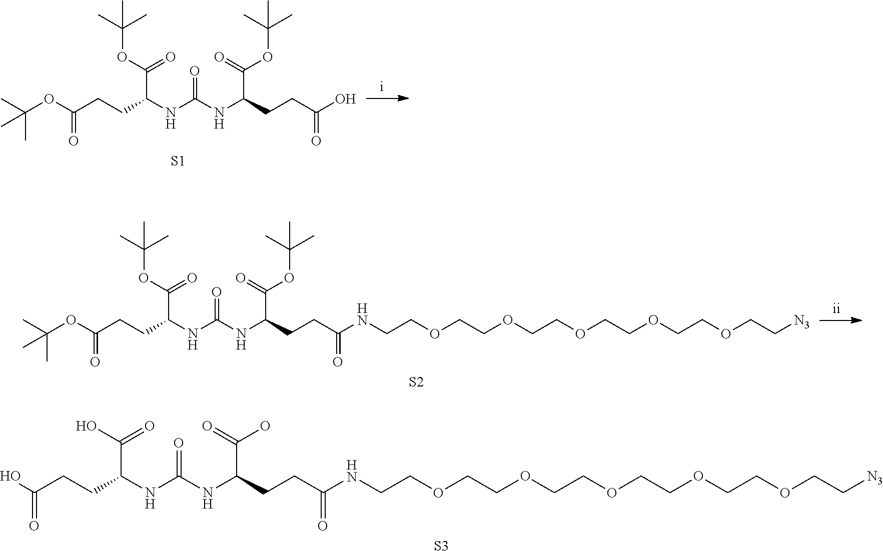 [SMILES: CC(C)(C)OC(=O)CC[C@@H](NC(=O)N[C@H](CCC(=O)O)C(=O)OC(C)(C)C)C(=O)OC(C)(C)C ; "S1" ; CC(C)(C)OC(=O)CC[C@@H](NC(=O)N[C@H](CCC(=O)NCCOCCOCCOCCOCCOCCN=[N+]=[N-])C(=O)OC(C)(C)C)C(=O)OC(C)(C)C ; "S2" ; [N-]=[N+]=NCCOCCOCCOCCOCCOCCNC(=O)CC[C@@H](NC(=O)N[C@H](CCC(=O)O)C(=O)O)C(=O)O ; "S3"]

Specific operation steps:

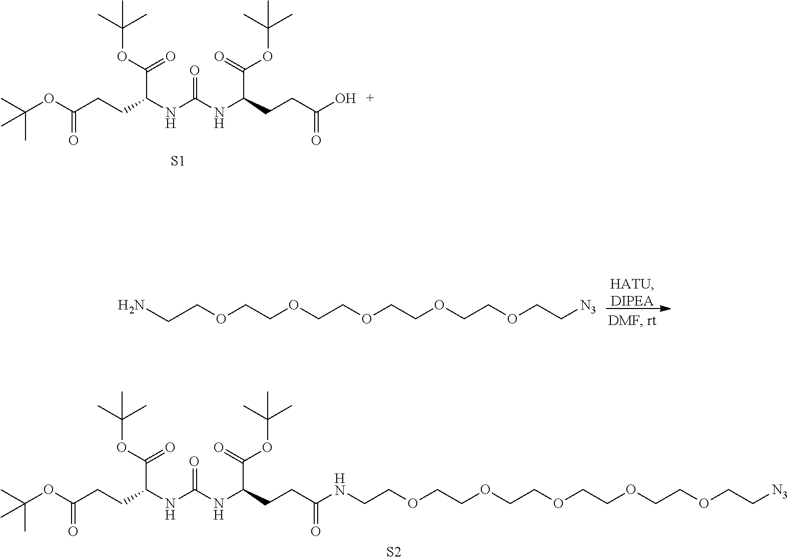

(i) Compound S1 (244 mg, 0.5 mmol) and 2-(7-aza-benzotriazol-1-yl)-N,N,N′,N′-tetramethylureinium hexafluorophosphate (HATU, 228 mg, 0.6 mmol) were added together into 5 mL of dimethylformamide. After addition of N,N-diisopropyl ethylamine (DIPEA, 129 mg, 1 mmol) under stirring conditions, the mixture was stirred for 15 minutes at room temperature. 17-azido-3,6,9,12,15-pentaoxaheptadecan-1-amine (182 mg, 0.6 mmol) was immediately added to the mixture as a spray and the reaction was continued for 6 hours at room temperature. At the end of the reaction, the reaction mixture was poured into 20 mL of water, extracted with ethyl acetate (50 mL×3), the organic phase was collected, washed with saturated salt water, dried with anhydrous sodium sulfate, and the solvent was removed under reduced pressure to obtain an orange oil crude product. The residue was separated and purified by column chromatography (ethyl acetate/petroleum ether=1/5) to obtain the yellow oil form compound S2 (338 mg, yield: 87%).

¹H NMR (400 MHZ, CDCl₃) δ6.93 (s, 1H), 5.67 (s, 2H), 4.28 (d, J=7.3 Hz, 2H), 3.66-3.50 (m, 20H), 3.48-3.30 (m, 4H), 2.30-1.96 (m, 6H), 1.89-1.72 (m, 2H), 1.47-1.29 (m, 27H); ¹³C NMR (101 MHZ, CDCl₃) δ172.81, 172.56, 172.28, 171.95, 157.49, 81.89, 81.66, 80.44, 70.64, 70.62, 70.55, 70.52, 70.48, 70.43, 70.42, 70.05, 69.98, 69.49, 53.02, 50.63, 39.15, 32.54, 31.63, 29.19, 28.18, 28.05, 27.98. HRMS (ESI): m/z [M+Na]⁺ calculated for C₃₅H₆₄N₆NaO₁₃⁺: 799.4424, found 799.4428.

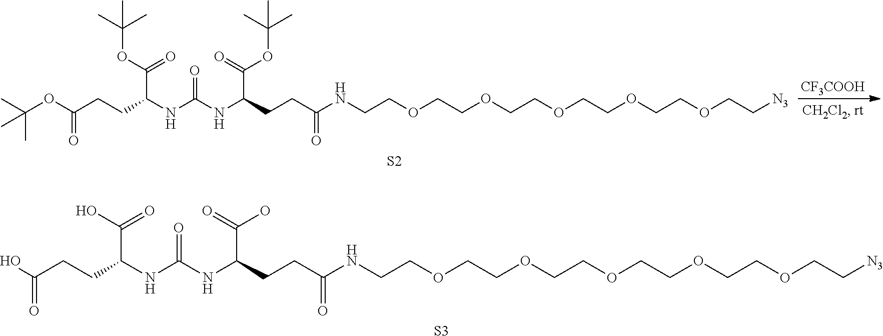 [SMILES: CC(C)(C)OC(=O)CC[C@@H](NC(=O)N[C@H](CCC(=O)NCCOCCOCCOCCOCCOCCN=[N+]=[N-])C(=O)OC(C)(C)C)C(=O)OC(C)(C)C ; "CF3COOH" ; "S2" ; [N-]=[N+]=NCCOCCOCCOCCOCCOCCNC(=O)CC[C@@H](NC(=O)N[C@H](CCC(=O)O)C(=O)O)C(=O)O ; "S3"]

(ii) Compound S2 (472 mg, 0.5 mmol) was dissolved in an equal volume mixed solution (5 mL) of dichloromethane and trifluoroacetic acid under ice bath conditions, and the reaction solution was left to react for 6 hours at room temperature. The solvent was removed by evaporation, and the resulting residue was purified by rapid column chromatography (MeOH/CH2Cl2=1/3) to obtain compound S3 in oil form (238 mg, 98%).

¹H NMR (400 MHz, DMSO-d₆) δ12.52 (s, 3H), 7.94 (d, J=9.2 Hz, 2H), 6.36 (dd, J =7.9, 5.6 Hz, 2H), 4.16-3.98 (m, 2H), 3.63-3.58 (m, 2H), 3.57-3.50 (m, 14H), 3.42-3.35 (m, 5H), 3.18 (q, J=5.9 Hz, 2H), 2.29-2.19 (m, 2H), 2.14-2.08 (m, 2H), 1.95-1.84 (m, 2H), 1.77-1.65 (m, 2H); ¹³CNMR (101 MHz, DMSO) δ174.77, 174.67, 174.27, 171.83, 162.78, 157.73, 70.28, 70.24, 70.16, 70.14, 69.72, 55.40, 52.58, 52.15, 50.43, 38.97, 36.25, 32.00, 31.23, 30.42, 28.71, 28.09. HRMS (ESI): m/z [M−H]⁻ calculated for C₂₃H₃₉N₆O₁₃: 607.2581; found: 607.2578.

### Preparation Example 3

This preparation example is used to illustrate the synthesis of 3′-terminal propargyl-modified small nucleic acids

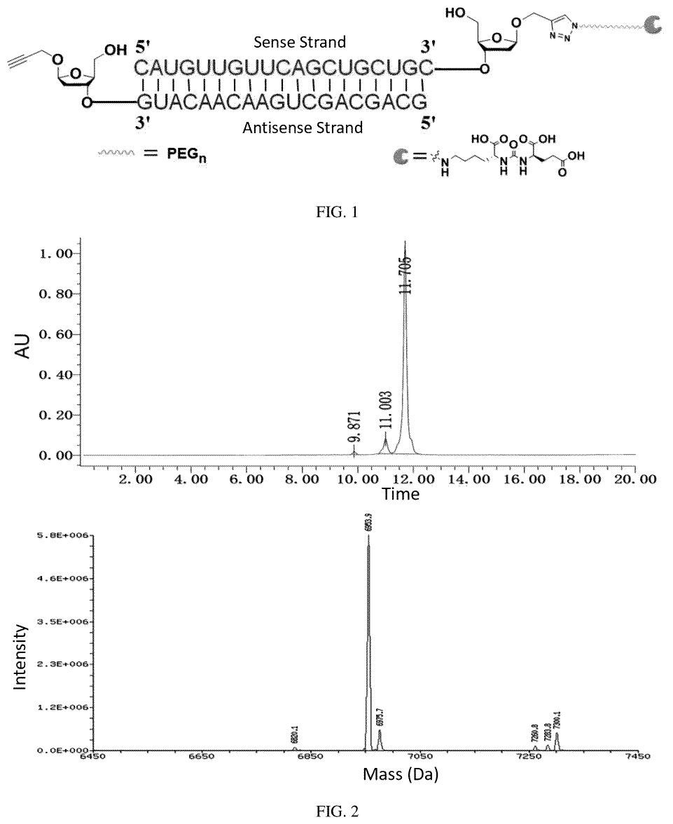

1-O-propargyl-2-deoxy-D-furanose (X, i.e. propargyl monomer), as a universal overhang modification, was attached to the 3′-terminal of small nucleic acids (oligonucleates) by using an ABI 394 DNA/RNA synthesizer (ABI, USA) (as shown in FIG. 1).

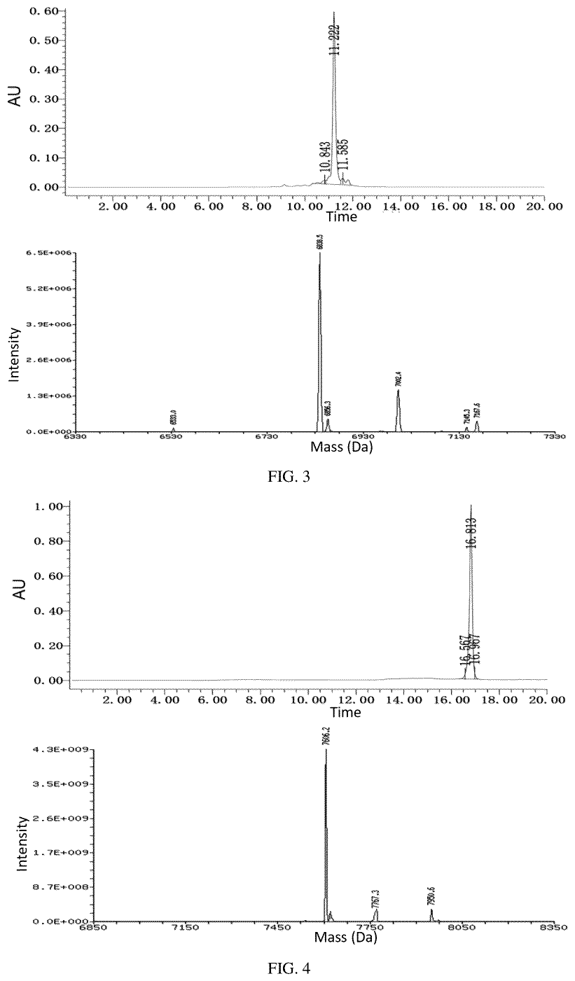

The specific steps of synthesis of 3′-terminal propargyl-modified small nucleic acids are as follows: firstly, 1-O-propargyl-2-deoxy-D-furanose phosphoramidite monomer was synthesized from commercial Hoffer's chlorosugar (Bioorg. Med. Chem., 2013, 5583-5588); then, the resulting phosphoramidite monomer was linked to a controlled pore glass (CPG), and linked to the 3′-terminal of the propargyl monomer X oligonucleotide by the automated nucleic acid synthesizer described above; finally, the CPG and the protective group were removed to obtain different 3′-terminal propargyl-modified single-stranded small nucleic acids. The purity of all propargyl-modified single-stranded nucleic acids prepared by the present disclosure was confirmed by high performance liquid chromatography and the structure was confirmed by mass spectrometer (FIG. 2-4).

TABLE 1
Different 3′-terminal propargyl-modified
single-stranded small nucleic acids

| ONS | | |
|---|---|---|
| No. | Sequence (SS/AS) | |
| ON1 | 5′Cy5-CAUGUUGUUCAGCUGCUGCUUX-3′ | SEQ ID NO: 1 |
| ON2 | 5′-CAUGUUGUUCAGCUGCUGCUUX-3′ | SEQ ID NO: 2 |
| ON3 | 5′-GCAGCAGCUGAACAACAUGUUX-3′ | SEQ ID NO: 3 |
| ON4 | 5′-GCGACGACCUUACAGAGCGUUX-3′ | SEQ ID NO: 4 |
| ON5 | 5′-CGCUCUGUAAGGUCGUCGCUUX-3′ | SEQ ID NO: 5 |

### Example 1

This example is used to illustrate the efficient synthesis of KUE-PEG-siRNA conjugates Synthesis of KUE-PEG2-siRNA conjugates: Propargyl-modified small nucleic acid sequence (ON3) (10 nmol), azido-modified targeting ligand 6a (30 nmol), and Cu(I)-TBTA (50 nmol) were successively added into a 200 μL sterile centrifugation tube to maintain the final concentration of RNA at about 100 μM. DMF (25% of the reaction liquid volume) was added to the system. The reaction system was mixed by gentle vortex and placed in a metal reactor, carrying out at 37° C. and under a shock condition of 900 r/min for 3 h. The reaction results were monitored by 15% denaturing polyacrylamide gel electrophoresis. Finally, the product was purified by the oligonucleotide extraction kit, and the purity and concentration of the product were confirmed by the NanoDrop 2000 ultramicrospectrophotometer (Thermo, USA). The final yield was 98%.

### Examples 2-12

It was done in the manner of Example 1, except that the type and/or amount of organic solvent added were varied. The results are shown in Table 2.

TABLE 2

| | | The ratio of the volume of | |
|---|---|---|---|
| | | organic solvent to the volume | yield |
| No. | Organic solvent | of the reaction liquid (%) | (%) |
| 1 | DMF | 25 | 98 |
| 2 | DMF | 5 | 58 |
| 3 | DMF | 50 | 90 |
| 4 | methyl alcohol | 5 | 50 |
| 5 | methyl alcohol | 25 | 78 |
| 6 | methyl alcohol | 50 | 65 |
| 7 | ethyl alcohol | 5 | 48 |
| 8 | ethyl alcohol | 25 | 82 |
| 9 | ethyl alcohol | 50 | 62 |
| 10 | acetone | 5 | 43 |
| 11 | acetone | 25 | 68 |
| 12 | acetone | 50 | 52 |

### Examples 13-20

It was done in the manner of Example 1, except that the type and/or amount of catalyst were varied. The experimental results are shown in Table 3.

TABLE 3

| | | Molar ratio of catalyst to | yield |
|---|---|---|---|
| No. | copper catalyst | small nucleic acid sequence | (%) |
| 13 | Cu(I)-TBTA | 1.5 | 62 |
| 14 | Cu(I)-TBTA | 3 | 88 |
| 15 | CuCl | 1.5 | 40 |
| 16 | CuCl | 3 | 75 |
| 17 | CuCl | 5 | 89 |
| 18 | CuBr | 1.5 | 42 |
| 19 | CuBr | 3 | 77 |
| 20 | CuBr | 5 | 86 |

### Example 21

It was done in the manner of Example 1, except that 6a was replaced by an equimolar amount of azido-modified targeting ligand 6b. The final yield was 96%.

### Example 22

It was done in the manner of Example 1, except that 6a was replaced by an equimolar amount of azido-modified targeting ligand 6c. The final yield was 95%.

### Example 23

This example is used to illustrate the efficient synthesis of KUE-PEG-siRNA conjugates targeting different genes.

It was performed in the manner of Example 1, except that RNA targeting the STAT3 gene (ON3) was replaced by RNA targeting the PHB-1 gene (ON5). The final yield was 97%.

### Example 24

This example is used to illustrate the efficient synthesis of DUPA-PEG-siRNA conjugates It was done in the manner of Example 1, except that 6a was replaced by an equimolar amount of azide-modified DUPA ligand. The final yield was 98%.

### Example 25

The single-stranded conjugates obtained from Examples 1-22 and the Cy5-labeled complementary strand small nucleic acids were incubated at a molar ratio of 1:1 in annealing buffer (2 mM magnesium acetate containing magnesium ions) at 95° C. for 3 minutes to form different KUE-PEG-siRNA conjugates and DUPA-PEG-siRNA conjugates.

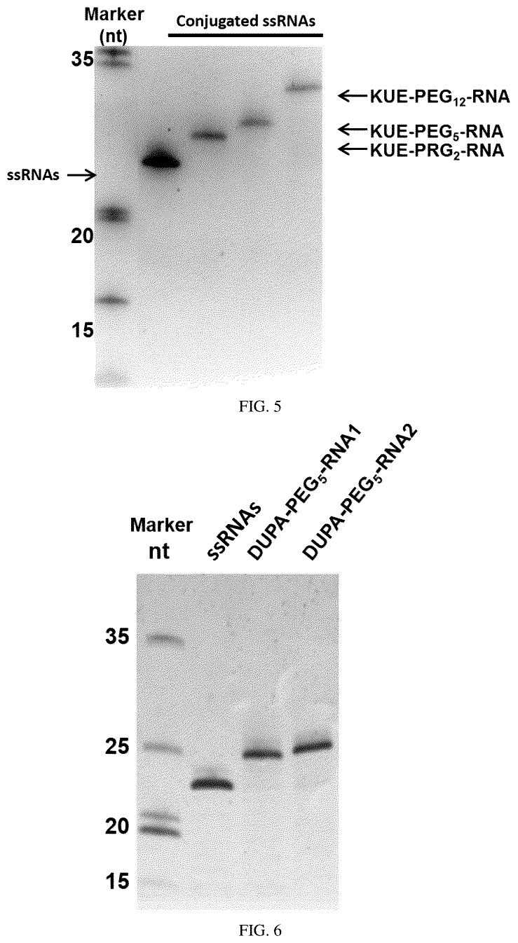

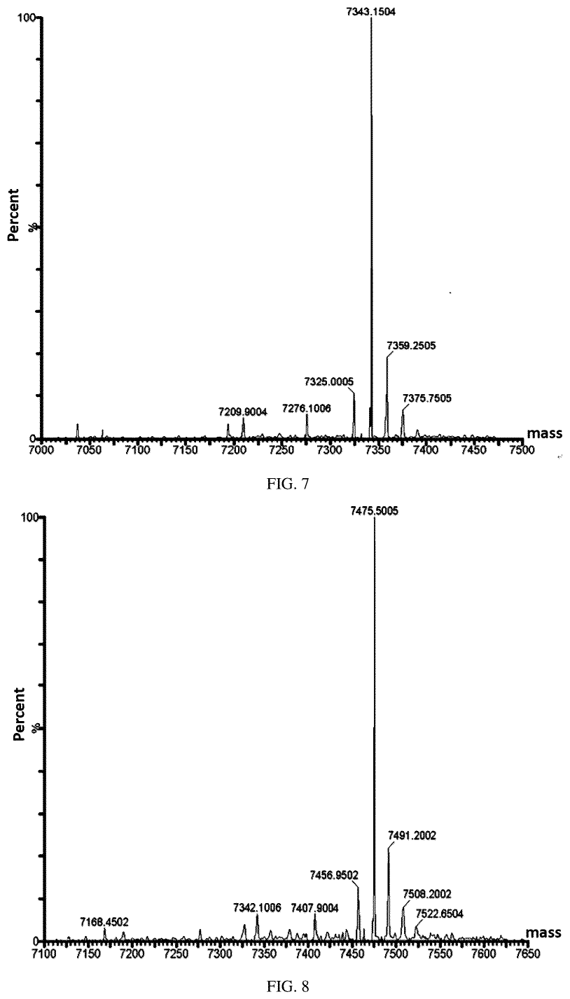

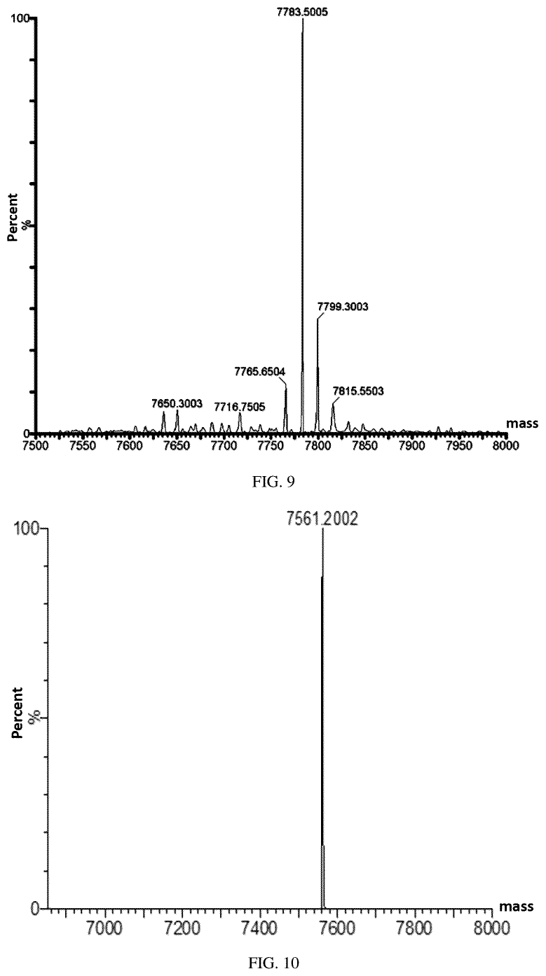

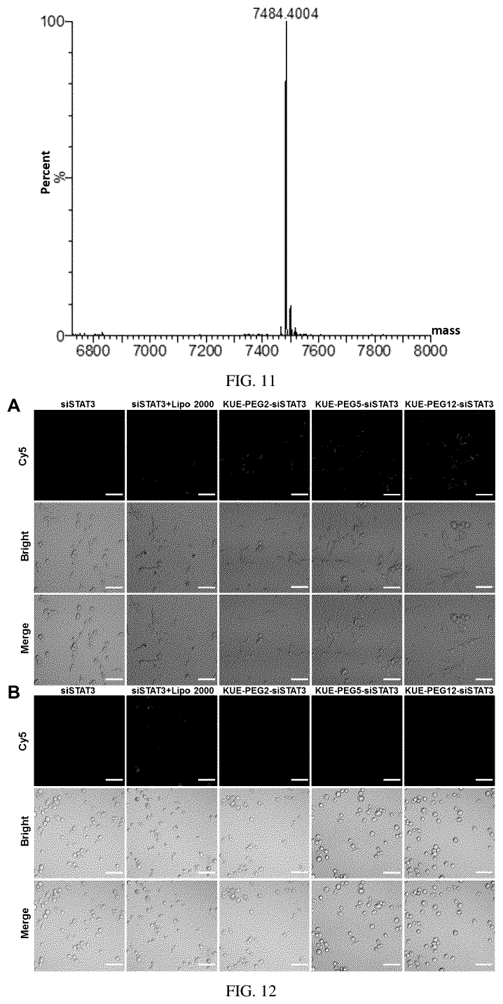

Sign diagrams of polyacrylamide gel electrophoresis results of the nucleic acid conjugate KUE-PEG2-siRNA, nucleic acid conjugate KUE-PEG5-siRNA, nucleic acid conjugate KUE-PEG12-siRNA, and DUPA-PEG5-siRNA obtained from Examples 1, 21, 22, 23, and 24 are shown in FIG. 5 and FIG. 6; Mass spectrogram are shown in FIGS. 7, 8, 9, 10 (Example 23) and 11 (Example 24), respectively.

### Test Example 1

### Selectivity Evaluation of KUE-PEG-siRNA Conjugates for Uptake in Prostate Cancer Cells

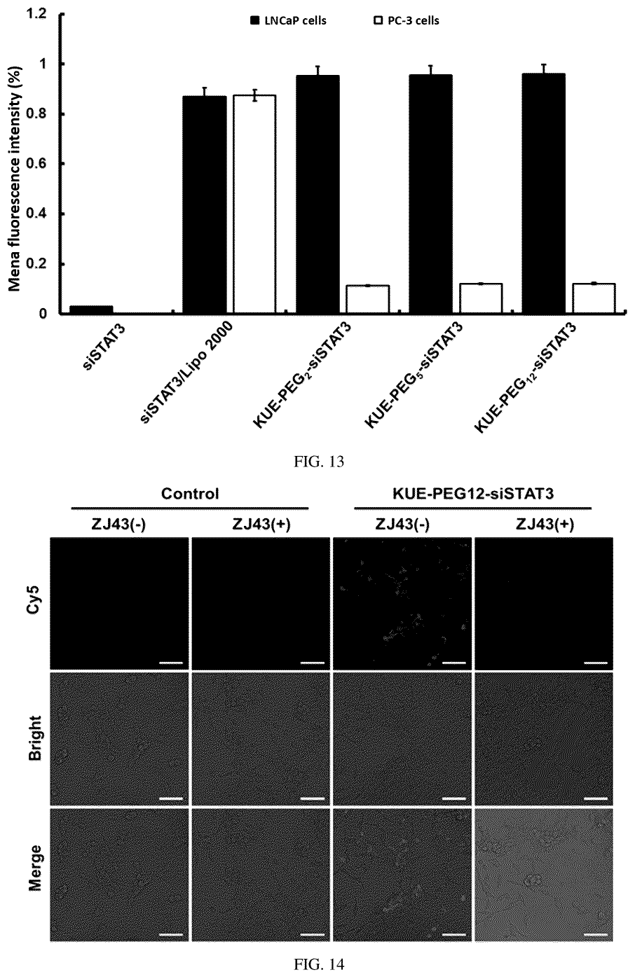

Cell imaging: LNCaP cells (PSMA-positive, purchased from Chinese Academy of Sciences) and PC3 cells (PSMA-negative, purchased from Chinese Academy of Sciences) were selected to evaluate the cellular uptake of KUE-PEG-siRNA conjugates. Cy5-labeled KUE-PEG-siRNA conjugates and siRNA (50 nM) were co-incubated with both prostate cancer cells, and cells were fluorescently imaged at 649 nm excitation light conditions 6 h after transfection (FIG. 12A (fluorescence imaging of LNCaP cells) and FIG. 12B (fluorescence imaging of PC3 cells)). From FIG. 12A, it could be found that red fluorescent signals from the siRNA group were only observed in the presence of lipofectamine 2000 (Invitrogen, Thermo Fisher) in both types of cells, whereas red fluorescent signals from the KUE-PEG-siRNA conjugate group could be observed in LNCaP cells. In addition, in FIG. 12B, the red fluorescence signal from the KUE-PEG-siRNA conjugate group was barely observable in PC-3 cells. These results suggest that KUE-PEG-siRNA conjugates, unlike unconjugated siRNAs, are able to selectively enter prostate cancer cells and that this internalization can be accomplished with high efficiency without the help of lipofectamine 2000. The average fluorescence intensity statistics of the cell fluorescence imaging in FIG. 12A (see FIG. 13) showed that the average fluorescence intensity of the KUE-PEG-siRNA conjugate group in LNCaP cells was 95%, while that of the siRNA group transfected by lipofectamine 2000 was 82%; and the mean fluorescence intensity of KUE-PEG-siRNA conjugates in PC-3 cells was less than 5%. These results indicated that siRNAs could not enter almost any cells without the aid of transfection reagents, whereas KUE-PEG-siRNA conjugates were able to selectively enter LNCaP cells without the aid of transfection reagents.

Internalization: The pathway of KUE-PEG-siRNA conjugates into LNCaP cells was further confirmed by adding a PSMA-specific inhibitor (ZJ43, Eur. J. Neurosci., 2004, 483-494). Wherein, LNCaP cells were co-incubated with ZJ43 ahead of time, followed by addition of Cy5-labeled KUE-PEG-siRNA conjugates. At 6 h, LNCaP cells were subjected to fluorescence imaging under 640 nm excitation light conditions (see FIG. 14). The results in FIG. 14 suggested that the addition of ZJ43 made the red fluorescence signal from KUE-PEG-siRNA conjugate almost undetectable in LNCaP cells, relative to the control group without PSMA specific inhibitor. This indicated that the addition of ZJ43 can significantly inhibite the uptake of KUE-PEG-siRNA conjugate by LNCaP cells, relative to the group without ZJ43 addition.

### Test Example 2

### Evaluation of Uptake Efficiency of KUE-PEG-siRNA Conjugates in Prostate Cancer Cells

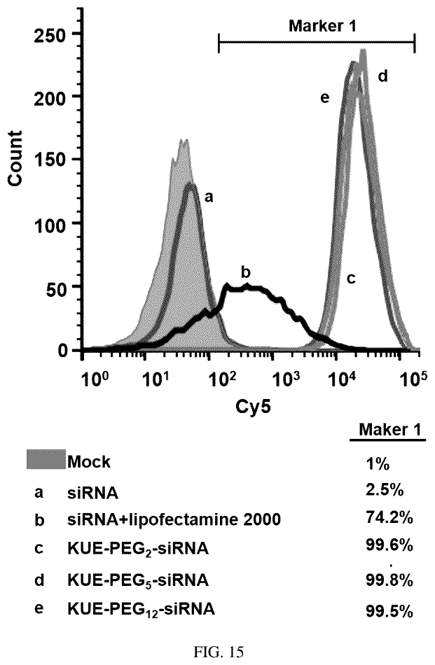

PSMA-positive expressing LNCaP cells (purchased from the Chinese Academy of Sciences) were selected to evaluate the cellular uptake efficiency of KUE-PEG-siRNA conjugates. Cy5-labeled KUE-PEG-siRNA conjugate (50 nM) and siRNA (50 nM) were co-incubated with LNCaP cells, wherein siRNA was co-transfected with lipofectamine 2000 (brand: Invitrogen, manufacturer: Thermo Fisher) as a control group. After 4 hours, the cells were treated according to the protocol of AnnexinV-FITC/PI apoptosis double staining kit (brand: 556547, manufacturer: BD Company, USA). The prepared cell samples were analyzed by FACS Calibur flow cytometer in the corresponding detection channel for Cy5. In the process of the experiment, the blank control group, siRNA transfection group, siRNA and lipofectamine 2000 co-transfection group, and different KUE-PEG-siRNA conjugate transfection groups were analyzed, and 10⁴ cells were collected and detected in each group. After the cell detection of all experimental groups was completed, the experimental results were analyzed using FlowJo software (FIG. 15). FIG. 15 shows that siRNA without ligand conjugation (siSTAT3) almost cannot enter LNCaP cells, which is consistent with the results of cell imaging. In addition, compared with the cell uptake efficiency of lipofectamine 2000 transfection group (74.2%), the KUE-siRNA conjugates (KUE-PEG2-siRNA, KUE-PEG5-siRNA, KUE-PEG12-siRNA) in LNCaP cells showed much higher cell uptake efficiency (>99%). These results imply that the conjugation of ligand KUE is indispensable for the delivery of siRNA into a specific prostate cancer cell line (LNCaP cells).

### Test Example 3

To verify the RNAi activity of the KUE-PEG-siRNA conjugate, a therapeutic small nucleic acid targeting the STAT3 gene was used in this test example, the sequence of which was shown in Table 4.

TABLE 4

| ONs | | |
|---|---|---|
| No. | sequence (SS/AS) | |
| ON2 | 5′-CAUGUUGUUCAGCUGCUGCUUX-3′ | SEQ ID NO: 2 |

Propargyl-modified small nucleic acid sequence (ON2) (10 nmol), azido-modified targeting ligand (30 nmol), and Cu(I)-TBTA (50 nmol) were successively added to a 200 μL sterile centrifugation tube to maintain the final RNA concentration at about 100 μM. DMF (25% volume of reaction liquid) was added to the system. The reaction system was mixed by gently vortex and placed in a metal reactor, carrying out at 37° C. and under a shock condition of 900 r/min for 3 hours. Finally, the reaction results were monitored by 15% denaturing polyacrylamide gel electrophoresis, and the single-stranded conjugates were isolated and purified by an oligonucleotide extraction kit. When the targeting ligand 6a was used, the yield was 97.7%; when the targeting ligand 6b was used, the yield was 96.2%; when targeting ligand 6c was used, the yield was 95.6%.

The resulting single-stranded conjugate and complementary strand small nucleic acid were then incubated at a molar ratio of 1:1 in annealing buffer (2 mM magnesium acetate) at 95° C. for 3 minutes to form KUE-PEG-siSTAT3 conjugates.

1) Evaluation of gene silencing effect of KUE-PEG-siSTAT3 conjugate:

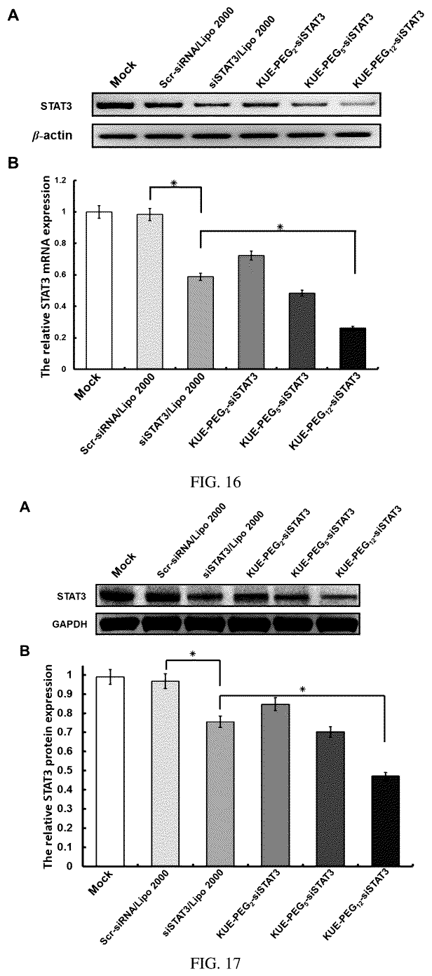

Target gene silencing: KUE-PEG-siSTAT3 (KUE-PEG-siRNA) conjugates (100 nM) targeting signal transduction and activator of transcription 3 (STAT3) were co-incubated with LNCaP cells. After 48 hours, mRNA expression was measured by reverse transcriptase-polymerase chain reaction (RT-PCR). As shown in FIG. 16A, all KUE-PEG-siRNA conjugates were able to effectively reduce STAT3 mRNA expression, wherein KUE-PEG5-siSTAT3 showed comparable silencing activity to siRNA transfected with the aid of lipofectamine 2000. In addition, it can be seen from FIG. 16B that the mRNA expression gradually decreased when the PEG length in the conjugate increased. These results indicated that KUE-PEG-siSTAT3 conjugate could effectively reduce the expression of the targeted gene after uptaking by LNCaP cells. Moreover, the conjugate KUE-PEG₁₂-siSTAT3 induced stronger mRNA down-regulation than KUE-PEG₂-siSTAT3 and KUE-PEG₅-siSTAT3.

Inhibition of protein expression: In addition to mRNA down-regulation experiments, the effects of the conjugates on STAT3 protein expression was explored by Western blotting (Western Blot) (see FIG. 17A and FIG. 17B). The results in FIG. 17A showed that the gray scale of the corresponding bands of all KUE-PEG-siSTAT3 conjugate experimental groups was reduced to varying degrees compared with the unconjugated siSTAT3 group, indicating that KUE-PEG-siSTAT3 conjugate can inhibit the expression of STAT3 protein. The results of quantitative experiments in FIG. 17B showed that KUE-PEG₁₂-siSTAT3 conjugate down-regulates STAT3 protein expression to the greatest extent.

2) Evaluation of the growth inhibition effect of KUE-PEG-siRNA conjugate in prostate cancer cells

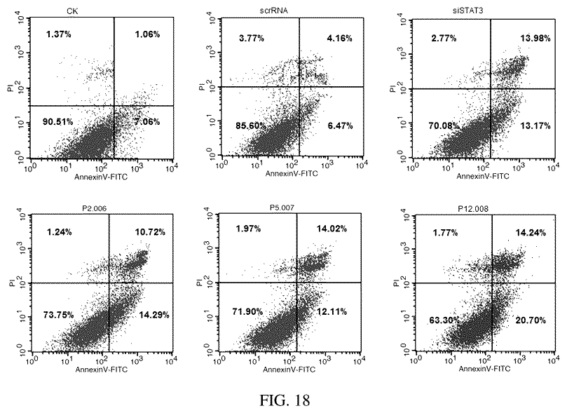

Based on the biochemical evaluation above, it was determined that KUE-PEG-siSTAT3 (KUE-PEG-siRNA) conjugate could be specifically taken up by prostate cancer cells LNCaP and induce the RNAi mechanism leading to significant reduction of STAT3 mRNA and protein expression. The effect of KUE-PEG-siSTAT3 conjugate on the growth of LNCaP cells was detected by flow cytometer (manufactor: BD, USA, model: FACS Calibur) (see FIG. 18). In FIG. 18, flow cytometry results showed that the KUE-PEG-siSTAT3 conjugates targeting STAT3 gene could promote the apoptosis of LNCaP cells to varying degrees (25.01%-34.94%), and the conjugate KUE-PEG₁₂-SiSTAT3 (KUE-PEG₁₂-SIRNA) could promote apoptpsis in 34.94% of LNCaP cells.

It can be seen from the above that the KUE-PEG-siSTAT3 conjugates constructed in the present disclosure can be selectively taken up by prostate cancer cells (LNCaP cells), and can trigger the RNAi mechanism in LNCaP cells to finally successfully inhibit the growth of prostate cancer cells.

Furthermore, further studies showed that the conjugates of the present disclosure were comparable to conventional means (e.g. liposomes) of delivering small nucleic acids in reducing the expression of target genes (or inhibiting the growth of cancer cells).

Preferred embodiments of the disclosure are described in detail above, however, the disclosure is not limited thereto. Within the scope of the technical conception of the present disclosure, a variety of simple variants of the technical scheme of the present disclosure can be carried out, including the combination of each technical feature in any other suitable way. These simple variants and combinations should also be regarded as the contents disclosed in the present disclosure and belong to the scope of protection of the present disclosure.

1-11. (canceled)

12. A conjugate formed by an azido-modified targeting ligand covalently linked to a propargyl-modified small nucleic acid sequence.

13. The conjugate of claim 11, wherein the azido group in the azido-modified targeting ligand is covalently linked to the targeting ligand by at least one fragment of polyethylene glycol.

14. The conjugate of claim 13, wherein the targeting ligand is a dipeptide formed by two amino acids in which at least one amino acid is glutamic acid; wherein,
the targeting ligand is able to recognize and bind to antigens expressed on the surface of target cells through glutamate; the antigen is PSMA;
and/or, the target cell is selected from at least one of prostate cancer cells, neuronal cells, renal cancer cells, and colon cancer cells.

15. The conjugate of claim 14, wherein the amino acids are selected from lysine and glutamic acid, glutamic acid and glutamic acid, and glutamic acid and glutamate analogs.

16. The conjugate of claim 12, wherein the azido-modified targeting ligand has a structure as follows: 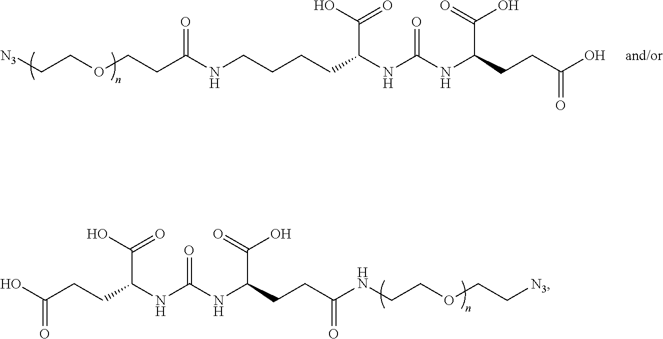
wherein n is an integer from 1 to 100.

17. The conjugate of claim 12, wherein the small nucleic acid sequence is selected from at least one of the small nucleic acids targeting STAT3, PHB1, Notch1, PLK1 and BRD4;
and/or, the propargyl modification is a 3′-terminal propargyl modification;
and/or, the propargyl-modified small nucleic acid sequence is modified to the 3′-terminal of the small nucleic acid sequence using the phosphoramidite method by solid phase synthesis technology; wherein, the propargyl compound contains at least one active hydroxyl group.

18. The conjugate of claim 17, wherein the propargyl compound has a structure as follows: 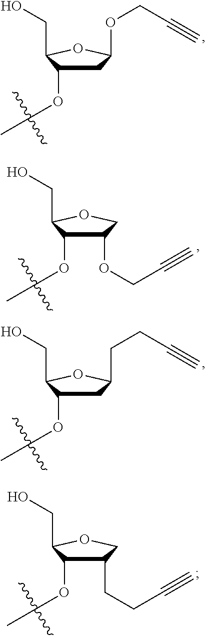
wherein, [symbol] represents the small nucleic acid ligation site.

19. A method for preparing a conjugate, comprising: making contact between an azido-modified targeting ligand and a propargyl-modified small nucleic acid sequence in the presence of a copper monovalent catalyst.

20. The method of claim 19, wherein
the molar ratio of the azido-modified targeting ligand to the propargyl-modified small nucleic acid sequence is (1.05-10): 1;
and/or, the targeting ligand is a dipeptide formed by two amino acids in which at least one amino acid is glutamic acid;
and/or, the azido-modified targeting ligand has a structure as follows: 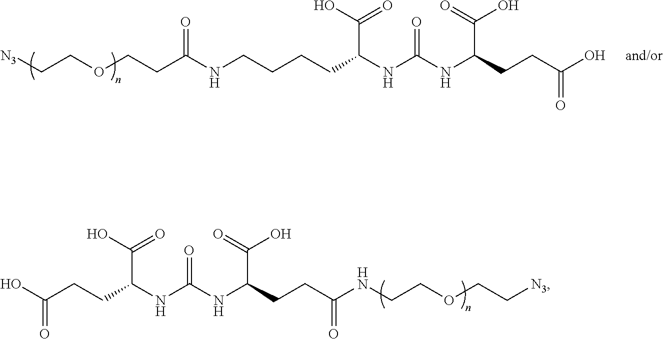
wherein n is an integer from 1 to 100;
and/or, the small nucleic acid sequence is selected from at least one of the small nucleic acids targeting STAT3, PHB1, Notch1, PLK1 and BRD4;
and/or, the propargyl modification is a 3′-terminal propargyl modification;
and/or, the propargyl-modified small nucleic acid sequence is modified to the 3′-terminal of the small nucleic acid sequence using the phosphoramidite method by solid phase synthesis technology.

21. The method of claim 20, wherein the molar ratio of the azido-modified targeting ligand to the propargyl-modified small nucleic acid sequence is (2-4): 1;
and/or, the amino acids are selected from lysine and glutamic acid, glutamic acid and glutamic acid, and glutamic acid and glutamate analogs;
and/or, the propargyl compound has a structure as follows: 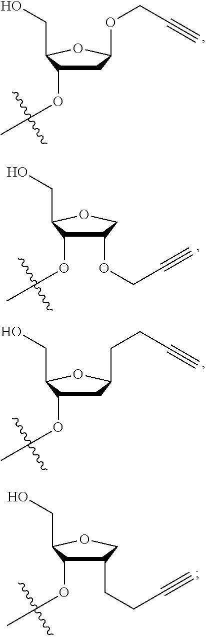
wherein, [symbol] represents the small nucleic acid ligation site.

22. The method of claim 19, wherein the copper monovalent catalyst is selected from at least one of Cu(I)-TBTA, CuBr and CuCl;
and/or, relative to 1 mol of a small nucleic acid sequence, the amount of the copper monovalent catalyst is 2-10 mol;
and/or, the conditions for the contact comprising: temperature of 35.5-38.5° C.; time of 1-5 h.

23. The method of claim 22, wherein the copper monovalent catalyst is Cu(I)-TBTA;
and/or, relative to 1 mol of a small nucleic acid sequence, the amount of the copper monovalent catalyst is 3-6 mol;
and/or, the conditions for the contact comprising: temperature of 36.5-37.5° C.; time of 2-4 h.

24. The method of claim 19, wherein the contact is carried out in the presence of an organic solvent selected from at least one of N,N-dimethylformamide, methanol, ethanol, and acetone.

25. The method of claim 19, wherein the small nucleic acid sequence is a siRNA sequence comprising a sense strand and an antisense strand, and the method comprise:
(1) propargyl-modified sense strand and propargyl-modified antisense strand were obtained respectively;
(2) single-stranded conjugates were obtained by contacting the azide-modified target ligand with the propargyl-modified sense strand or propargyl-modified antisense strand;
(3) the conjugate was obtained by incubating the single-stranded conjugate with the other strand in annealing buffer at 90-100° C. for 1-5 minutes.

26. The method of claim 15, wherein the annealing buffer is selected from a magnesium acetate solution of 1.5-2.5 mM.

27. A method for treating a disease related with an abnormality in a tissue expressing PSMA, comprising: administering a therapeutically effective amount of the conjugate of claim 12 to a subject with the disease.

28. A method for treating a disease related with an abnormality in a tissue expressing PSMA, comprising: administering a therapeutically effective amount of the conjugate prepared by the method of claim 19 to a subject with the disease.

29. The method of claim 27, wherein the disease related with an abnormality in a tissue expressing PSMA is occurred in glandular tissue, colon, kidney and nervous system; wherein, the glandular tissue is selected from one of the prostate, pancreas, breast and thymus.
